(19) United States
(12) Patent Application Publication
(10) Pub. No.: US 2006/0116355 A1
(43) Pub. Date: Jun. 1, 2006

(54) Eye care kit for treating periocular disease

(76) Inventor: Eduard T. Van Breen, Manchester, VT (US)

Correspondence Address: Arthur R Eglington, 601 Stoney Run Road, R.D. #5, Pottsville, PA, 17901, US

(21) Appl. No.: 10/997,000

(22) Filed: Nov. 26, 2004

(51) Int. Cl. A61K 31/685; A61K 31/66; A61K 31/4172; A61K 31/19; A61M 35/00

(52) U.S. Cl. 514/78; 514/143; 514/400; 514/574; 604/1

(57) ABSTRACT

A care compliance kit is revealed to minimize and prevent the spreading of highly contagious bacterial and viral ocular diseases, to improve conditions for healing, to optimize the functioning of topical antibiotics and to advance compliance to a two-step system approach for effective eye hygiene for Blepharitis, Conjunctivitis, and Dry Eye patients. The compliance kit consist of housing to accommodate at least one method for presoaking the hardened crusts and waxy debris in and around the eyelashes, at least one lid cleansing medium to remove the debris from the eyelashes and eyelids, a container to secure the grouping of products and instructions. Preferably, the eyelash-soaking medium is presented in a single use, unilateral (one eye only), disposable, pre-moistened, sterile, soft medium containing a nonirritating solution, ideally suited to dissolve the hardened debris, the eyelid cleansing solution is composed of a nonocular irritating surfactant also individually presented to be used in one time use, unilateral disposable manner. Instructions are enclosed detailing how to apply the soaking and cleansing solutions.

### CROSS REFERENCE TO OTHER APPLICATIONS

None.

### FIELD OF INVENTION

The present invention generally relates to eye care kits. More specifically the invention relates to minimizing and prevention of the spreading of highly contagious and chronic bacterial and viral ocular infections, using the reagents of a multi-component eye care kit, in facilitating a stringent compliance to a critical two-step eye hygiene paradigm.

### BACKGROUND OF THE INVENTION

Bacterial infections (followed by viral infection and allergies) are the main sources for two of the most common eye infections and inflammations, (Conjunctivitis and Blepharitis) and are causal to the world's primary eye disease “Dry Eye”, affecting both infants and adults. Either highly contagious or chronic in nature said inflammations cause excess tearing and discharge, which accumulate and harden on the eyelashes and eyelid margins

Conjunctivitis is an inflammation or infection of the conjunctiva, the membrane lining the eyelids that helps keep the eyes moist. The most common form, Infectious Conjunctivitis is highly contagious and could spread rapidly including cross infection from one eye to the other eye (unilateral to bilateral), re-infecting within the environment such as household, school, or office. It is mainly spread through contaminated fingers or fomites (contaminated objects) and articles such as washcloths.

Signs of a conjunctival condition include flaky, waxy debris and hardened crust of discharge on the eyelashes and lid margins.

Blepharitis is also one of the most common eye diseases throughout the world. It also affects people of all ages. Blepharitis is a generic term for several types of eyelid inflammation usually surrounding the lid margin and eyelashes. The condition is rather difficult to manage; while generally not contagious, it is however mostly chronic, with no known cure. If left untreated certain types of Blepharitis may lead to infection, eyelid scarring, scarring of the cornea and loss of vision.

Blepharitis manifests itself through scaling and flaking around the eyelashes, excess sebum production and oily scaly discharge, mucopurulent discharge, and matted, hard crusts around the lashes. The crust, discharge or debris accumulating on the eyelashes and lid margins form an ideal environment for the indigenous Staphylococci bacteria, naturally found on the skin of the eyelids further increasing the chance of infection, allergic reaction and tear break down. Blepharitis disturbs the production of the critical lipid (outer) layer of the tear film which causes the entire tear to evaporate, resulting in Dry Eye (Dry Eye is the world's number one eye disease. It is described, as pandemic and epidemic with no know cure) A reduced tear flow does not contain sufficient enzymes and antibodies to kill bacteria. In addition, a reduced tear quantity doesn't properly dilute bacteria and irritants, nor wash inflammatory products away from the lashes and lid margin, so they accumulate and create further inflammation worsening the cycle of disease, with Blepharitis, Meibomian Gland Dysfunction and Dry Eye perpetuating each other.

Chronic Bacterial Conjunctivitis and Blepharoconjuntivitis.

The terms underscore that Conjunctivitis often develops in association with Blepharitis and visa versa, but also that misdiagnosis is possible because the symptoms of the sticky mucopurulent, hardened matting of the lashes are similar. This mass of inflamed granulation tissue and debris may in turn harbor and fester additional organisms (bacteria, fungi, acid fast bacteria) or foreign matter

Treatment:

In all acute cases, it is critical to bring symptoms under control as soon as possible and thorough eye hygiene is prescribed as the primary and prolonged treatment. A two-step eye hygiene method is suggested as many times as needed in the acute stages and at least twice per day. Practicing proper eye hygiene can prevent conjunctivitis and control and maintain Blepharitis and any topically applied ointment can only best penetrate the skin and function optimally, without the hardened crusts.

Currently mostly household, non-sterile warm washcloths with baby shampoo are employed. Such washcloths often have a very high count of agents, such as bacteria or fungi, that can actually cause diseases. Again the “cure” has become part of the problem.

Causes for non-compliance to the critical first soaking step are multiple, but most often it is the lack of easy availability. There is often a need to administer the two-step eye hygiene therapy multiple times per day, especially in the acute stages. Outside the home, while shampoo or eyelid hygiene products may be available, soaking mediums or warm washcloths are not. Washcloths should never be shared to avoid spreading of the highly contagious diseases. Thus the critical soaking and softening part of the treatment is either delayed, or eyelid hygiene is administered without the first step, thus further perpetuating a vicious circle causing a cascade of events including and ranging from unnecessary prolonged discomfort, spreading of highly contagious infections, worsening of chronic symptoms, to ultimately permanent eye damage and blindness.

U.S. Pat. No. 6,112,900 discloses a different kind of eye care kit aimed at contact lens wearers wherein different combinations of contact lens products and solutions (such as contact lens wetting and soaking solution, contact lens cleansing solution, contact lens storing solution, contact lens storage case, housing and instructions) are provided in combination with an eyelid cleanser. The eyelid cleanser is focused on contact lens wearers and the kit omits a soaking and softening method and instructions.

### SUMMARY OF THE INVENTION

According to the present invention, the fist critical step is warm soaking and softening of the waxy and matted hardened crusts on the eyelashes and eyelid margins in order to avoid trauma of the affected area caused by a too strenuous cleansing-and-rubbing action, which would lead to small, bleeding and oozing ulcers, worsening the condition of the patient. The warmth also causes blood vessels to dilate improving blood flow and healing, and importantly, the heat opens infected gland pores and ducts to allow for a most thorough cleansing of affected area.

The invention facilitates a thorough removal of dried, hardened and greasy secretions and crusts on the eyelashes and lid margins associated with Conjunctivitis, Blepharitis, Dry Eye and other ocular conditions that require strict eye hygiene, and optimizes the application, penetration and functioning of topically applied ointments to the affected areas. Most specifically the invention describes a portable, disposable, single use, unilateral (each eye) eye care kit and instructions that facilitates compliance to a two-step method, whereby the second step, the removal and cleansing of hardened and waxy debris on the eyelashes and lid margins is expressly employed only after a critical first step, which is comprised of soaking and softening of said debris, to facilitate an improved and meticulous eye hygiene paradigm.

In a preferred embodiment of the invention, the compliance kit comprises the soaking medium whichis sterile and wherein the cleansing solution further contains: a pH-control agent, citric acid (1.50); a first water soluble, surfactant potassium C12-13 monoalkyl phosphate (5.0); a second water soluble, surfactant disodium lauroamphodiacetate (4.0); a preservative diazolidimyl urea propylene (1.00); a skin conditioning agent phospholipid essential fatty acid (0.1-5.0); and a viscosity regulating agent sodium chloride (7.00).

More specifically as to the compliance kit, the non-irritating cleansing solution contains deionized water, PEG-80, Sorbitan Laurate, Sodium Trideceth Sulfate, Cocamidopropyl Betaine, Sodium Lauroamphoacetate, PEG-150 Distearate, Sodium Laureth-13 Carboxylate, Sodium Chloride, Citric Acid, Boric Acid, Cocoamidopropyl PG-Dimonium Chloride Phosphate, Sodium Hydroxymethylglycinate, Disodium EDTA, and Oxtoxynol-9

### BRIEF DESCRIPTION OF THE DRAWINGS

FIG. 1 shows a schematic of a eye hygiene compliance kit according to the present invention, composed of four main components;

FIG. 2 shows the compliance kit of FIG. 1 in which the eye cleansing solution is contained in either a vial or a foaming element;

FIG. 3 shows the compliance kit of FIG. 1 in which the cleansing solution component is impregnated in a Q-tipped rod;

FIG. 4 shows the compliance kit of FIG. 1 in which the three care components are deposited under a flexible film;

FIG. 5 shows the compliance kit of FIG. 4 in which only the printed instructions component are mounted external of the flexible film cover area;

FIG. 6 shows the compliance kit of FIG. 3 in which all three components are deposited under flexible film area;

FIG. 7 shows the compliance kit of FIG. 1 now in which the soaking solution is contained in either a vial, coupled with application by pad, or in a spraying device

FIG. 8 shows the compliance kit of FIG. 7 in which all the components are deposited and secured under a flexible film area;

FIG. 9 is a multiple units kit of any the components kits of FIG. 1-8, in which each care solution component is aligned linearly with transverse perforation lines, forming a ribbon of plural containers, along with a set of instructions component;

FIG. 10 is a schematic of a dispenser container in which the free end of the package ribbon of kits FIG. 9 are extended from the container and are adapted for selective separation along the perforations as a complete compliance such kit as depicted in FIG. 1;

FIG. 11 is a schematic of FIG. 1 in which the soaking solution component and the instructions component are the same but the cleansing solution component can be seen in the form of a Q-tip, or be contained in a liquid vial, or impregnated in a foamable carrier; and

FIG. 12 is a schematic of the kit of FIG. 11 in which all the kit components are deposited and secured under a flexible film area for preservation and sterility.

### DETAILED DESCRIPTION OF THE DRAWINGS

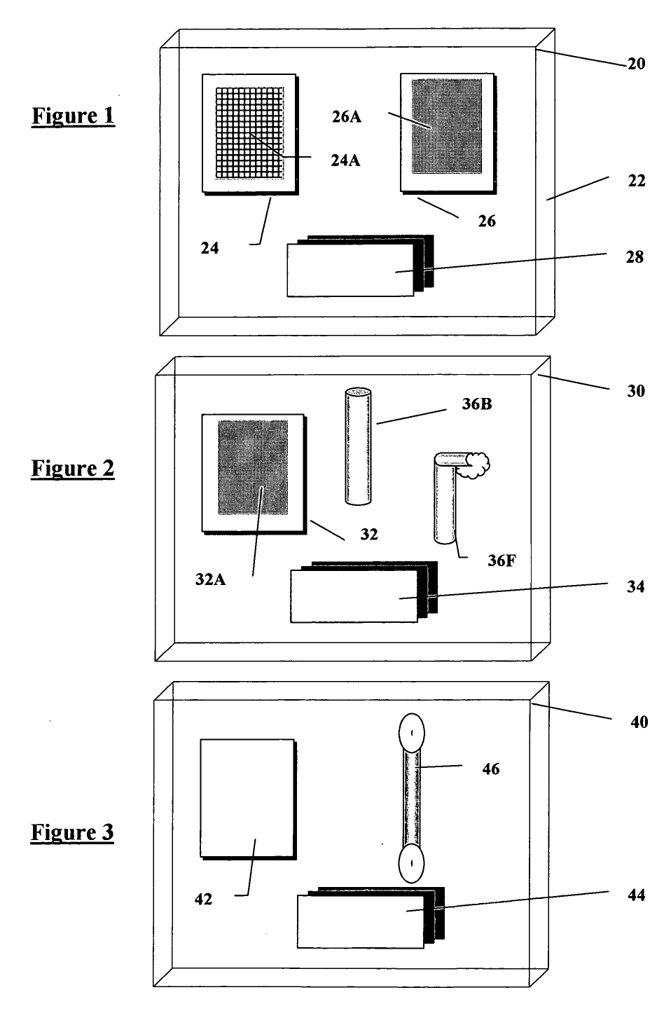

Referring now to the drawing, and to FIG. 1 in particular, which depicts a first embodiment of the eye care kit 20 of this invention. The kit comprises a housing 22 for nesting and securing the other functional components of the kit. These other components comprise: a medium 24 containing an aqueous soaking solution adapted for removal of hardened crust and waxy debris in and around the eyelashes and eyelid margins; at least one non-irritating, aqueous cleansing solution 26, adapted to cleanse and remove from the eyelids the duly softened encrustation and debris build up. The cleansing solution basically comprises a pH-control agent, at least one water soluble, non-irritant surfactant, a preservative, a skin conditioning agent, and a viscosity regulating agent. Preferred reagents for each of these elements are set forth later and in the working example as well.

As to the soaking solution composition, this is preferably an aqueous medium in which a mild mineral salt is employed, preferably sodium chloride. In one embodiment the soaking solution is contained as impregnated in a disposable gauze-like fabric 24A enveloped in an impervious wrapper and the cleansing solution is impregnated in a separate disposable pad 26A enveloped in a separate impervious wrapper.

Instructional means 28 are also included in the kit. They serve for informing the patients of the need for a two-step approach to eye hygiene: firstly, to soften and soak the waxy and hardened crusts and debris present on the eyelashes and periocular skin; and, secondly, to effectively remove and cleanse such crust and debris from the periocular area for improved eye health. The specific text for the instructional component is well within the competence of professional oculists, like optometrists and ophthalmologists to draft.

In one embodiment, housing 22 is a box, as a shown in FIGS. 1 to 5. Preferably, such a housing is a cardboard container preferably with a transparent front panel. The kit operating components, 24, 26, and 28, can be secured within this type of housing, or in an alternative types, such as a plastic bubble container unit, for example, variations of which are within the teachings of the invention.

In a preferred kit, housing 22 and components 24, 26, and 28, can all be sized for the convenience of the user. The optimal size of housing 22, which is suitable for a travel kit or starter kit, is within the range of two inches to about four inches in height; about three inches to six inches in width, and about two inches to four inches in depth. The solution containers can be sized to hold reagents within a range of 2 cc to 5 cc in liquid volume. The instruction sheets are of standard printing.

In FIG. 2, an alternative care kit, 30, is shown. A soaking solution 32 is contained as impregnated in a disposable gauze-like fabric 32A enveloped in an impervious wrapper, primarily to maintain sterility; and the instructions 34 are enclosed, but set apart, just as in the embodiment of FIG. 1. However, the other medium containing the cleansing solution 36 is contained either in a vial 36B or in a foam-forming device 36F. Capped reagent vials are conventional in the industry, calling for no further elaboration, and they come in varied sizes to meet specific needs. As noted, the bottled form of cleansing solution can be in a vial or other plastic container. The foam-forming device is capable of generating a transiently stable foam from said cleansing solution in the form of a controlled concentration foam, suitable for direct application to an eyelid of a subject.

In FIG. 3, another embodiment of the care kit 40 is shown, which includes the liquid soaking component 42, and the standard instruction sheet component 44, while the cleansing solution is novelly contained in a cotton-tipped rod 46 (Q-Tip), which has been pre-saturated with the cleansing solution. The manner of use of the Q-Tip to cleanse the pretreated (by soaking) periocular area is within the skills of the kit user upon reading the instructional component 44.

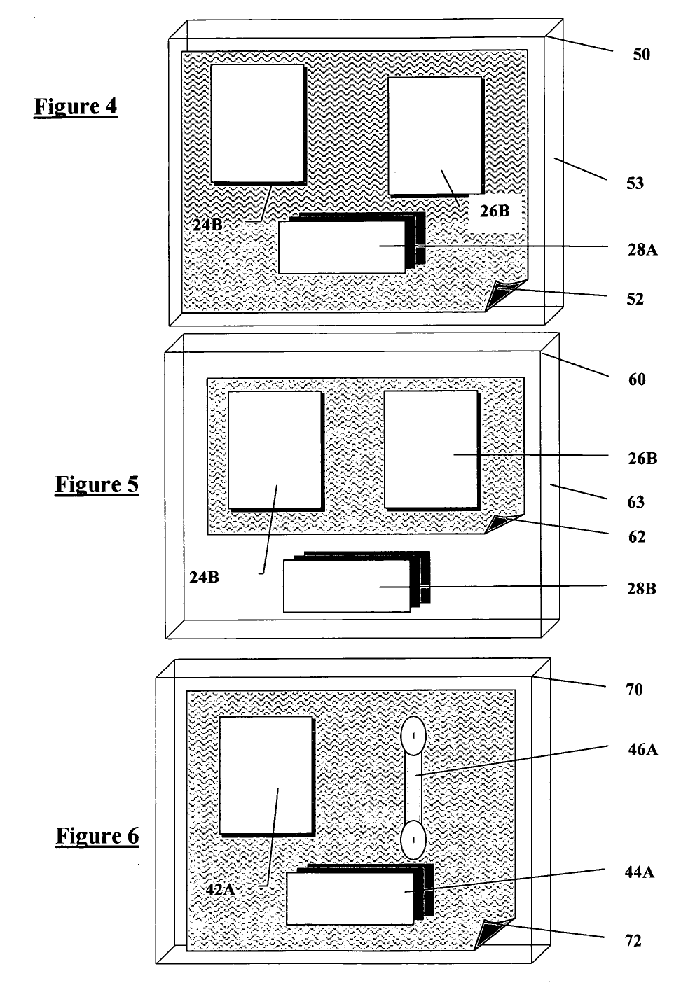

In the kit 50 of FIG. 4, the three operative components: 24B, 26B, and 28A, are identical to those of the embodiment shown in FIG. 1, but all are now secured under an optionally transparent flexible film 52, which film is readily separable from the support kit 50, upon a slight manual effort to access components.

An alternative kit array 60 of the active components of FIG. 4 is depicted in FIG. 5 (24B, 26B, and 28B), wherein the instruction component 28B is disposed externally of the flexible film sheet 62; but is secured to the kit 60; this serves to facilitate contents perusal prior to separation of the packets of active reagents.

In the embodiment of FIG. 6, the operative components are identical to those of FIG. 3, except they are now all disposed under a separably, flexible film 72.

In the kit embodiment 80 of FIG. 7, the soaking solution is either contained in the form of a vial 72V, or loaded in a pre-saturated pad 72P (kept sterile). One alternative is to enclose the soaking solution in a spraying element, such as 72S. The cleansing solution 82 and the instruction sheet 84 are disposed, just as depicted in embodiment of FIG. 1.

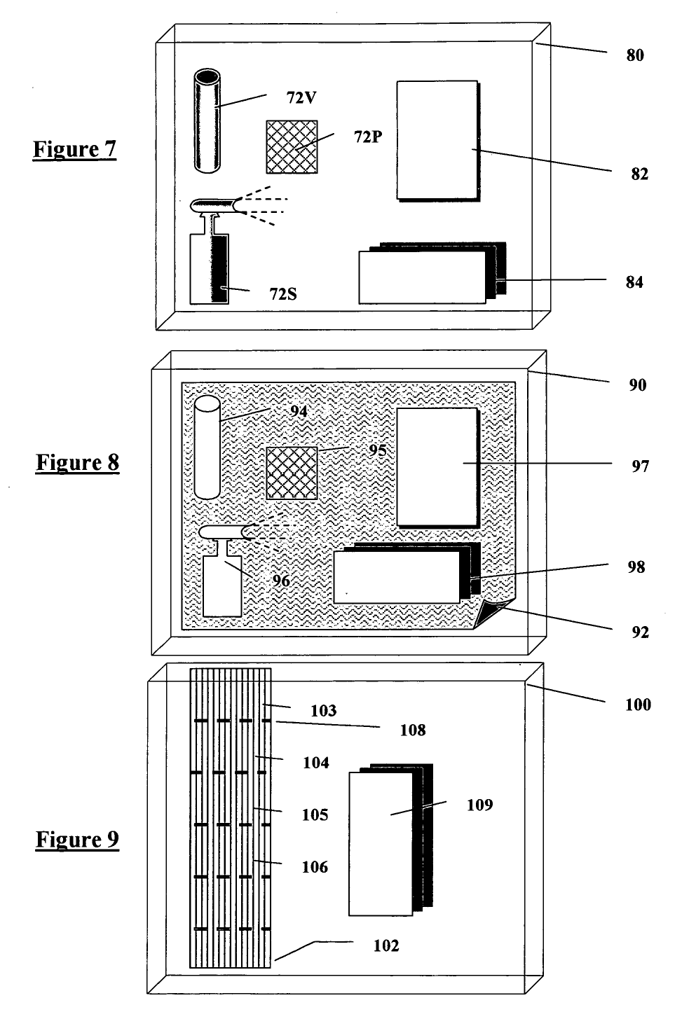

The kit embodiment 90 of FIG. 8 is the same as that of FIG. 7, save that a flexible film 92 overlays almost all of the housing surface, but holding in place all of the operative components, 94, 95, 96, and 97, including the instructions 98 there under.

The dispensing article 100 of FIG. 9 shows schematically how the useful solutions of the present invention can be configured in a continuous ribbon form 102, whereby each of the containers 103 to 107 has a physical connection to the next single container, thus forming a ribbon 102 of plural containers. This is fabricated with the connection between each such single container being perforated line 108, to facilitate their manual separation. The instructions component 109 is separately secured along the ribbon 102.

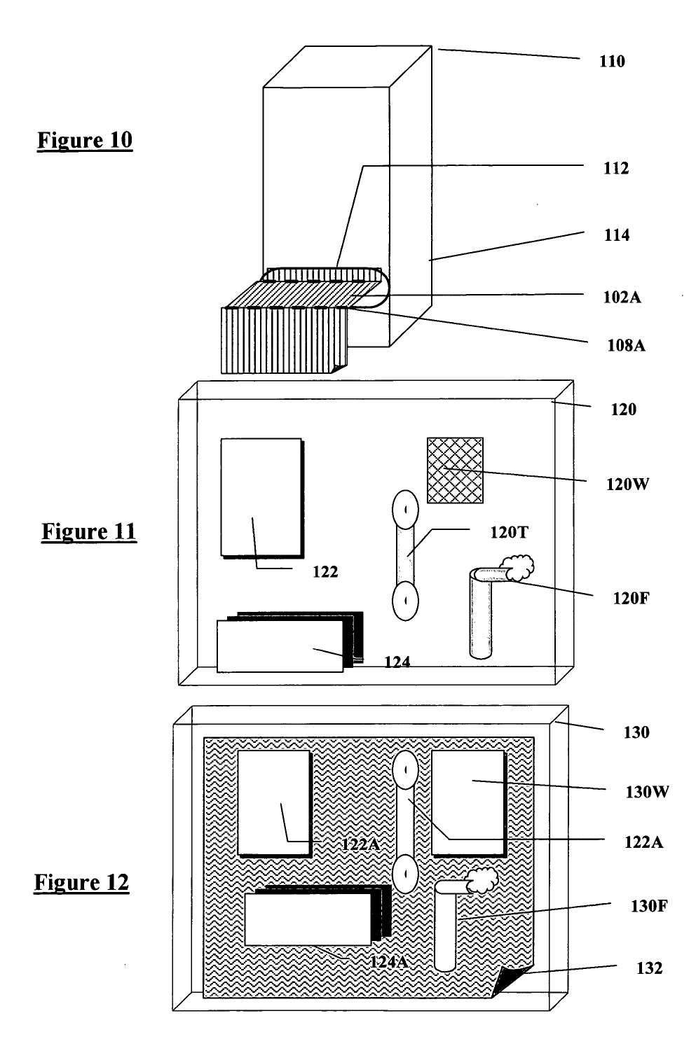

In the device of FIG. 10, the ribbon-like form of the kit components are preloaded into a box-like carton 110, also provided with a transverse slot 112, located proximal to the lower front edge of the carton 114. This serves to dispense kit units like that of FIG. 8 as needed for treatment from a convenient multi-kit bearing article. [claim 15]

In the kit embodiment 120 of FIG. 11, the option is seen wherein the cleansing solution is presented in more than one container, each depicted container being the combination of at least one single wrapper 120W, with a cotton-tipped container 120T, and one forming device 120F. The soaking solution is 122 and instructions are 124.

In the kit embodiment 130 of FIG. 12, all the components are the same as depicted in FIG. 11, except that they are deposited under a flexible film 132.

### DESCRIPTION OF A PRESENTLY PREFERRED EMBODIMENT

The present invention utilizes surfactant compositions not irritating to the eye yet with an acid pH close to the natural acid pH of the dermis to minimize disruption of the protective acid mantle against pathogens of the skin. Among the commercially available water-soluble surfactants useful with this invention is a composition comprised of PEG-80, sorbitan Laurate, Sodium Trideceth Sulfate, Cocamidopropyl Betaine, Sodium Lauroamphoacetate, Peg-150 Distearate, Sodium Laureth-13 Carboxylate (Coladet BSB).

In another preferred embodiment of the invention the surfactant system useful with this invention are: sodium lauryl sulfate (CALFOAM ES 303); potassium C12-13 monoalkyl phosphate 60 (ARLATONE MAP 230-T60); C12-14 monoakyl phosphate (ARLATONE MAP 230K40); disodium lauroamphodiacetate (MONATERIC 949-J); and linoleamidopropyl PG-chloride phosphate (PHOSPHOLIPID EFA). Special ingredients such as Allantoin, Panthenol and Citric Acid are encapsulated in the cleansing solution so that the skin is in better shape after washing than before.

In yet another embodiment the invention employs a surfactant that includes HEPES derivatives which are pharmacologically active as anti-phospholipase and anti-inflammatory compounds specifically, wheren the active ingredients are certain long chain esters of selected zwitterionic compounds, based on an N-substituted taurine, namely aliphatic esters of HEPES. Generally, HEPES, and its homologs are N-(2-hydroxy ethyl)-piperazine-N′-(2-alkane sulfonic acid).

Generally, an alkali metal salt of HEPES is catalytically reacted with an alkyl-substituted, either saturated or unsaturated, aliphatic salt, such as methyl oleate, methyl linoleate, methyl palmitate, methyl stearate, methyl myristate, and methyl behenate. They are reacted in equimolecular amounts, carried out either with or without a non-aqueous solvent, such as acetone, and in a temperature range of 0° C. to variable degrees C.; which is between 0° C. and the chosen solvent's reflux temperature. The purification of the crude ester is carried out by means of crystallization in an organic solvent, dissolved in methanol, and recrystallized. The U.S. Pat. No. 6,114,337, granted Sep. 5, 2000, provides detailed synthesis examples of the zwitterionic organic compounds useful here, including the ether analog and urethane derivatives of HEPES.

### WORKING EXAMPLE 1

A soaking solution of the present invention comprises an alkaline metal salt, preferably sodium chloride present in a range of 0.6 to 6% by weight.

The cleansing solution of the present invention in a preferred embodiment comprises:
(a) Deionized water in a concentration range of 20-80%;
(b) Sodium Laureth Sulfate in a concentration range of 0.3-20% (CALFOAM ES 303);
(c) (c) Potassium C12-13 Monoalkyl Phosphate Polysorbate 60 in concentration range of 0.5-15% (ADLATONE MAP 230-T60);
(d) Disodium Lauroamphodiacetate in a concentration range of 0.02-12% (MONOTERIC 949-J);
(e) Linoleicamidopropyl PG-Diammonium Chloride Phosphate in a concentration range of 0.01%-5.0% (PHOSPHOLIPID EFA);
(f) HEPES Acetate in concentration range of 0.01-7.7%;
(g) Allantoin [Ed to supply the concentration range];
(h) H-panthenol [Ed to supply the range];
(i) Citric acid c. 1.50%.
   The manner of formulation of the above recited reagents for an ocular cleansing solution is within the skill of the art and does not call for repetition here.

What is claimed:

1. An infection control and eye hygiene compliance kit for the removal of hardened crusts and waxy debris in and around the eyelashes and eyelid margins, the kit comprising:
(a) at least one soaking solution adapted to soak and soften hardened encrusted eyelashes and infected eyelids is presented in first carrier medium, wherein said medium is saturated with an isotonic solution;
(b) at least one non-irritating cleansing solution adapted to cleanse and remove from the eyelashes and eyelids any softened encrustation and debris build-up, said cleansing solution comprising a pH-control agent; at least one water soluble, non irritant surfactant, a preservative; a skin conditioning agent, and a viscosity regulating agent;
(c) instructions for informing patients of the-need for a two-step system approach firstly to soak and soften the waxy and hardened crust and debris on the lashes and periocular skin, and, secondly how to properly remove and cleanse such crust and debris from the eyelashes and eyelids for improved eye health; and
(d) a housing for securing an aggregation of the soaking solution, the cleansing solution for the eyelashes and eyelids, and of the instructions on how to properly soak and cleanse the periocular area.

2. A compliance kit of claim 1 wherein the soaking medium is sterile and wherein the cleansing solution further contains: a pH-control agent, citric acid (1.50); a first water soluble, surfactant potassium C12-13 monoalkyl phosphate (5.0); a second water soluble, surfactant disodium lauroamphodiacetate (4.0); a preservative diazolidimyl urea propylene (1.00); a skin conditioning agent phospholipid essential fatty acid (0.1-5.0); and a viscosity regulating agent sodium chloride (7.00).

3. A compliance kit of claim 1 wherein the non-irritating cleansing solution contains deionized water, PEG-80, Sorbitan Laurate, Sodium Trideceth Sulfate, Cocamidopropyl Betaine, Sodium Lauroamphoacetate, PEG-150 Distearate, Sodium Laureth-13 Carboxylate, Sodium Chloride, Citric Acid, Boric Acid,, Cocoamidopropyl PG-Dimonium Chloride Phosphate, Sodium Hydroxymethylglycinate, Disodium EDTA, and Oxtoxynol-9

4. A compliance kit of claim 1 wherein the cleansing solution further contains HEPES acetate, panthenol, and allantoin,

5. An infection control and eye hygiene compliance kit for the removal of hardened crusts and waxy debris in and around the eyelashes and eyelid margins, the kit comprising:
(a) at least one soaking solution adapted to soak and soften hardened encrusted eyelashes and infected eyelids, wherein the said solution is saturated with an isotonic solution, and is enveloped in an impervious wrapper;
(b) a cleansing solution containing deionized water, in a concentration range of 20-80%, Sodium Laureth Sulfate in a concentration range of 0.3-20%, Potassium C12-13 Monoalkyl Phosphate Polysorbate 60 in concentration range of 0.5-15%, Disodium Lauroamphodiacetate in concentration range of 0.02-12%, Linoleamidopropyl PG-Diammonium Chloride Phosphate in concentration range of 0.01%-5.0%, Sodium Chloride in concentration range of 0.1-6%, HEPES Acetate in concentration range of 0.01-7.7%, Citric acid in concentration range of 0.5-7.5%, Diazolidinyl Urea Propylene Glycol/Methyl-Propyl Parabens in concentration range of 0.01-5.7%, panthenol in concentration range of 0.01-10%, Glyoxylic-Diureide/allantoin in concentration range of 0.01-15%), Polysorbate 80 in a concentration range of 0.01-5% and a fragrance in a concentration range of 0.005-3%, with all the stated ranges being by volume;
(c) instructions for informing patients of the need for a two-step system approach firstly to soak and soften the waxy and hardened crust, debris on the lashes and periocular skin and secondly how to properly remove and cleanse such crust and debris from the eyelashes and eyelids for improved eye health; and
(d) a housing for securing a combination of the soaking medium, the cleansing solution for the eyelashes and eyelids and the instructions on how to properly cleanse the periocular area.

6. The compliance kit of claim 1, wherein the soaking solution is present in a first carrier medium also adapted to soak and soften the encrusted eyelashes and eyelid margins, which first medium is enveloped in a first impervious wrapper, and wherein the cleansing solution is present in a second carrier medium adapted to remove, and cleanse the eyelids, with said second medium being separately enveloped in a second impervious wrapper.

7. The compliance kit of claim 1, whereby the cleansing solution is present in at least one of a bottle dispenser and of a foam-forming device.

8. The compliance kit of claim 1 whereby the cleansing solution is present in at least one cotton tipped rod, whereby the cotton tip rod is pre-filled with said cleansing solution, which can be released on demand.

9. The compliance kit of claim one wherein both of the soaking solution and the cleansing solution are enveloped in a single container, disposed within the housing, together with instructions how to apply the two step eye lid hygiene treatment, whereby there is at least one of soaking solution and cleansing solution in combination present within the housing.

10. The compliance kit of claim 9 wherein the instructions are enclosed externally of the single container and within the housing.

11. The compliance kit of claim 9 whereby the cleansing solution located in the single container is present in a pre-filled, cotton tipped applicator.

12. The compliance kit of claim 1 whereby the soaking solution is present in at least one container, such container to be selected from one of a bottle, a sterilized pad, and spraying device.

13. The compliance kit of claim 9 whereby as to the soaking solution present in the single container, the container is selected from one of a bottle, a pad, and a spraying device.

14. The compliance kit of claim 10 whereby the single container for the soaking solution and the cleansing solution are present in a ribbon form, whereby each single container has a physical connection to the next single container forming a ribbon of plural containers, with the connection between each single container being perforated to facilitate their separation.

15. The compliance kit of claim 14 whereby the housing of said ribbon includes a transverse slit so that one free end of said ribbon may project outwardly of the kit to facilitate easy access to a single container without the enter the housing.

16. The compliance kit of claim 1 whereby the cleansing solution is present in more than one container, each container being a combination of at least one or more of a single wrapper, with a cotton tip container and a one foaming devise.

17. A compliance kit of claim 9 whereby the cleansing solution is present in more than one container, with each container being a combination of at least one or more of a single wrapper, with a cotton tip container and one foaming device and are deposited under a flexible film.

18. A compliance kit of claim 1 whereby the soaking solution is comprised of sodium chloride.

19. An infection control and eye hygiene compliance kit for the removal of hardened crusts and waxy debris in and around the eyelashes and eyelid margins, the kit comprising:
(a) at least one soaking solution adapted to soak and soften hardened encrusted eyelashes and infected eyelids is presented in first carrier medium, wherein said first medium is saturated with an isotonic solution comprising sodium chloride;
(b) at least one non-irritating cleansing solution adapted to cleanse and remove from the eyelashes and eyelids any softened encrustation and debris build-up, said cleansing solution comprising a pH-control agent; at least one water soluble, non irritant surfactant, a preservative; a skin conditioning agent, and a viscosity regulating agent;
(c) instructions for informing patients of the need for a two-step system approach firstly to soak and soften the waxy and hardened crust and debris on the lashes and periocular skin, and, secondly how to properly remove and cleanse such crust and debris from the eyelashes and eyelids for improved eye health; and
(d) a housing for securing an aggregation of the soaking solution, the cleansing solution for the eyelashes and eyelids and the instructions on how to properly soak and cleanse the periocular area.

20. A compliance kit of claim 1 where to the aggregate of soaking and cleansing solutions is added a cotton tipped applicator.

21. A care kit for Conjunctivitis, Blepharitis and Dry Eye patients comprising at least one component for eyelash and eyelid soaking solution and at least one component of a cleansing solution, said cleansing solution being a gentle non irritating surfactant; instructions underscoring the importance of a two step eye hygiene paradigm; instructions how to administer the eyelash soaking solution, and how to administer the eyelash and eyelid cleansing solution; how to use said solutions in conjunction with each other, and the significance of single and unilateral methods to minimize cross infection; instructions also highlight the importance of immediately and properly disposing of the single use items to minimize chances that said items are used again by others and how to avoid spreading of contagious disease; said instructions are secured inside a housing which secures the eyelash soaking solution and the cleansing solution.

22. The kit of claim 19, whereby the instructions how to administer the two step, eye hygiene paradigm is detailed on the outside of the housing.
